(19) United States
(12) Patent Application Publication
(10) Pub. No.: US 2017/0260509 A1
(43) Pub. Date: Sep. 14, 2017

(54) Systems, Methods, and Devices for Cell Cycle Synchronization of Stem Cells

(71) Applicant: The Trustees of Columbia University in the City of New York, New York, NY (US)

(72) Inventors: Clark T. HUNG, Ardsley, NY (US); J. Chloë BULINSKI, Leonia, NJ (US); Andrea R. TAN, New York, NY (US)

(73) Assignee: The Trustees of Columbia University in the City of New York, New York, NY (US)

(21) Appl. No.: 15/600,428

(22) Filed: May 19, 2017

Related U.S. Application Data
(63) Continuation-in-part of application No. PCT/US2015/061624, filed on Nov. 19, 2015.
(60) Provisional application No. 62/081,904, filed on Nov. 19, 2014.

(51) Int. Cl. C12N 5/077

(52) U.S. Cl. CPC C12N 5/0655; C12N 2513/00; C12N 2501/15; C12N 2501/115; C12N 2501/135; C12N 2506/1346

(57) ABSTRACT

The present disclosure relates to methods and systems of tissue engineering, and, more particularly, to optimization of tissue regeneration using cell cycle synchronization of stem cells.

### CROSS-REFERENCE TO RELATED APPLICATIONS

This application is a continuation-in-part of international application PCT/US2015/061624 filed Nov. 19, 2015, which claims the benefit of U.S. Provisional Application No. 62/081,904 filed Nov. 19, 2014. Each of the above-identified applications is incorporated herein by reference in their entirety.

### STATEMENT REGARDING FEDERALLY SPONSORED RESEARCH

This invention was made with government support under AR046568, AR060361 and AR061988 awarded by the National Institutes of Health (NIH). The government has certain rights in the invention.

### FIELD

The present disclosure relates generally to tissue engineering, and, more particularly, to optimization of tissue regeneration using cell cycle synchronization of stem cells.

### BACKGROUND

There is a need to develop additional techniques and methodology for engineering tissue. To this end, the disclosure provides systems and methods of engineering tissue by utilizing cell cycle synchronization.

When tissue, such as cartilage, for example articular cartilage, has been damaged following injury or disease, cell-based strategies can be used to regain form and function. While stem cells can be developed as a clinical source, their promise is limited by their inefficient conversion to desired cell fates (toward cartilage, bone, fat, etc). Cell-cell variability is a major challenge to devising robust differentiation protocols for optimal replacement strategies after tissue has been damaged. Terminally-differentiated cells require reactivation of the cell cycle to generate requisite cell numbers, and then efficient re-establishment of phenotype.

Tissue repair strategies optimally use cell populations that uniformly undergo tissue-specific differentiation. Ideally, mesenchymal stem cells (MSCs) or patient-specific pluripotent stem (iPS) cells are presented with appropriate factors and physical cues to induce differentiation. However, heterogeneity is observed; only some of this can be due to mixed cell populations (contaminant cells). Instead, the varying responses of cells to differentiation cues may depend upon cell cycle phase.

### SUMMARY

In an aspect, the disclosure provides for methods of tissue processing, comprising
(a) synchronizing cell cycles of a plurality of cells,
(b) wherein the synchronizing comprises suspending the cells in an agent; and/or
(c) wherein the suspended cells are serum-starved.

In another aspect, the cells comprise, consist of, or consist essentially of stem cells. In another aspect, the stem cells are mesenchymal stem cells (MSCs) or patient-specific pluripotent stem (iPS) cells. In yet another aspect, the cells comprise undifferentiated cells.

In an aspect, the disclosure provides for methods of priming the cells in the S-phase of their respective cycles. In aspects, the disclosure provides for chemical priming and/or 3-D priming In another aspect, the priming is such that a uniformly differentiated progenitor cell population is formed.

In another aspect, the methods described herein provide for, prior to the synchronizing, removing terminally differentiated or progenitor cells from a patient. Methods described herein further provide for, after the removing and before the synchronizing, expanding the cells from the patient and optionally returning the cells to the patient and/or engineering a tissue graft.

The disclosure further provides for methods wherein the cell synchronizing comprises suspending cells in an agent and wherein the agent optionally comprises methylcellulose. In an aspect, the cells are suspended in an about 0.5% wt/vol, about 1% wt/vol, about 1.5% wt/vol, about 2% wt/vol, about 3% wt/vol, about 5% wt/vol methylcellulose solution, in an about 0.5% wt/vol to about 5% wt/vol, about 1% wt/vol to about 4% wt/vol, or about 1% wt/vol to about 2.5% wt/vol methylcellulose solution.

In another aspect, methods described herein provide for pelleting of S-phase synchronized cells so as to produce a homogeneous distribution across each pellet. The disclosure further provides for methods of engineering tissue, comprising
(a) synchronizing cell cycles of a plurality of cells,
(b) wherein the synchronizing comprises suspending the cells in an agent; and/or
(c) wherein the suspended cells are serum-starved, and
   wherein the engineered tissue is used in a tissue graft and/or returned to a patient.

In an aspect, methods described herein can include isolating or harvesting tissue from a patient and/or animal. In another aspect, the tissue is isolated or harvested tissue from, for example, stem cells, cartilage, bone marrow, human bone marrow, and cells derived from the synovial lining In yet another aspect, methods described are capable of priming stem cells such that they are differentiated to any particular type of tissue, for example, mesenchymal stem cells differentiated toward cartilage, bone, fat, and muscle.

In another aspect, the cells, for example, plurality of cells, are selected from the group consisting of stem cells, allogeneic cells, and autologous cells. In yet another aspect the stem cells are selected from the group consisting of mesenchymal stem cells, pluripotent stem cells, and embryonic stem cells.

### BRIEF DESCRIPTION OF DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements.

FIGS. 1A through 1C are images of the gross morphology of cells (FIG. 1A) pre-synchronization (asynchronous), (FIG. 1B) in methyl-cellulose suspension (G1 phase), and (FIG. 1C) related post synchronization (S-phase), where the inset is an image of immunofluorescent labeling of acetylated α-tubulin (green) with actin cytoskeleton (red) revealing non-homogeneous presentation (length, orientation) of primary cilium (white arrow), according to one or more embodiments of the disclosed subject matter.

FIGS. 2A through 2D are graphs of biosynthetic output of cell pellets at early (day 3) and late (day 42) timepoints for (FIG. 2A) Total GAG (retained by pellet and lost to media) and (FIGS. 2B-2D) biochemical compositions of pellets over time, wherein *p<0.05 versus control, according to one or more embodiments of the disclosed subject matter.

FIG. 3 are images of histological stains for cellularity (H&E), GAG (Safranin O), and collagen (Picrosirius Red) distribution for cell pellets derived from various cell cycle populations, where the illustrated scale bar represents 0.5 mm, according to one or more embodiments of the disclosed subject matter.

FIG. 4A is an image of alcian blue staining for glycosaminoglycan (GAG) in chondrocyte-seeded agarose constructs showing heterogeneity of cell elaborated matrix (day 7), where white arrow 402 points to a cell with little GAG and yellow arrow 404 points to a cell with rich GAG halo, according to one or more embodiments of the disclosed subject matter.

FIG. 4B is a schematic diagram illustrating the cell cycle, which can be modulated to optimize cell differentiation and matrix production, where G0: quiescent, G1: growth, and S: synthesis, according to one or more embodiments of the disclosed subject matter.

FIG. 5 is a schematic diagram illustrating aspects of cell synchronization, which can yield more efficient differentiation of cell populations as assessed in a physiological 3D culture (Ascaffold) to thereby produce more functional tissues, according to one or more embodiments of the disclosed subject matter.

FIG. 6 sets forth a representative cell cycle for (a) adult canine chondrocytes (total cell cycle time=43 hours) and (b) adult human mesenchymal stem cells (total cell cycle time=56.3 hours).

FIG. 7 sets forth representative histological stains for cellularity (H&E), GAG (Safranin O), and bulk collagen (Picrosirius Red) distribution for constructs comprised of asynchronous cells, serum starved cells, and serum recovery cells. Scalebar=0.5 mm.

FIG. 8 sets forth representative immunological stains for type I and type II collagen and aggrecan (Alexa Fluor 488 labeled) distribution for constructs comprised of asynchronous cells, serum starved cells, and serum recovery cells. Scalebar=0.5 mm.

FIG. 9 shows a representative Ca²⁺ signal in response to 0.5 dyne/cm² fluid shear stress over time with the dotted line indicating response threshold and downward arrows indicating the onset (120 sec.) and offset (230 sec.) of flow.

FIG. 10A shows a percentile response of cell mounting a calcium response for asynchronous and synchronized cells with n=100 cells, *p<0.05 vs an asynchronous group.

FIG. 10B shows primary cilia incidence (% of total cells).

FIG. 10C shows primary cilia length for control and PMC 27 cells with n=250 cells, *p<0.05 versus an asynchronous group.

FIG. 11 shows representative images that show the increased incidence and shorter primary cilia lengths for PMC 28 cells where Alexa-Fluor 488 conjugated acytle α-tubulin (indicated by arrow) is co-labeled with actin cytoskeleton (scaffold structure visible as streaky structure) and nucleus (ovoid blobs).

FIG. 12 shows normalized GAG/DNA over culture time for an example tissue engineering process according to embodiments of the disclosed subject matter.

FIG. 13 shows a linear regression of GAG output vs % S phase cells.

FIG. 14 shows a representative flow cytometry plot of % cell cycle phase.

FIGS. 15A and 15B are tables summarizing the representative flow cytometry data of FIG. 14.

FIG. 16 shows results of encapsulated the synchronized cells in agarose to create engineered cartilage constructs showing that for juvenile bovine chondrocytes, constructs with synchronized cells reach native properties in 2 weeks and for adult canine chondrocytes that have reduced biosynthetic activity, the ability to capture nature properties in as early as 28 days.

FIG. 17 shows an overview of examples and processes according to the disclosed subject matter.

FIG. 18 sets forth mechanical properties (Young's modulus (E_{Y}) and dynamic modulus (G*)) and GAG content for asynchronous and synchronized cell constructs from trial 1 and trial 2 of the study.

FIG. 19 sets forth normalized GAG/DNA biosynthetic output over 28 days in culture. *p<0.05 vs. asynchronous #p<0.05 vs. respective G1 synchronized state.

FIG. 20 shows biosynthesis output over culture time (n=3/5/group, *p<0.05 vs asynchronous group.

FIGS. 21A and 21B show linear regressions of biosynthetic output vs. % S phase cells (FIG. 21A) and % proliferating cells (FIG. 21B)

FIG. 22 shows representative histological stains for cellularity (H&E), GAG (Safranin O) and collagen (Picrosirius Red) distribution for cell pellets derived from various cell cycle populations at day 28 and GFP/tdTomato fluorescence stain for COL2A1 reporter expression at day 28 (Scalebar=100 micron).

FIG. 23 sets forth the Day 42 data of FIG. 19 (isolating serum groups).

### DESCRIPTION

In an aspect, the disclosure provides for methods of tissue processing by synchronizing cell cycles of a plurality of cells.

In one or more embodiments of the disclosed subject matter, a drug-free, bulk synchrony method can be used whereby undifferentiated stem cells and dedifferentiated chondrocytes can respond more uniformly and robustly to chemical and 3D priming in the S-phase of their cycle, subsequently producing superior tissue. In an aspect, the tissue is cartilage-like tissue. In an aspect, embodiments of the disclosed subject matter can use methylcellulose to synchronize chondrogenic precursors, and the chondrogenesis of synchronous populations of chondrogenic precursors can be examined Embodiments of the disclosed subject matter can be used, for example, to optimally differentiate MSCs and iPS cells for a number of tissue regeneration goals.

In an aspect, the disclosure provides for a method wherein cell populations in G1 to S phase described herein effect larger cuboidal cells than asynchronous populations. In another aspect, cell synchronization prior to subsequent 3D pellet formation alters the biosynthetic output and content of engineered cartilage. In another aspect, the disclosure provides for a method wherein total GAG retained by the pellet system is initially reduced by about 30%, about 40%, about 50%, about 60%, about 70%, or about 80% as early as day 1, 2, 3, 4, 5, or 10 in culture, reflecting altered biosynthetic activity. In another aspect, the disclosure provides for a method whereby in day 20, 25, 30, 35, 40, 42, 45, or 50 of culture, only S phase cells exhibit greater GAG content than asynchronous cells.

The disclosure further provides for a method wherein when GAG lost to the media is taken into account, the total GAG produced over 20, 25, 30, 35, 40, 42, 45, or 50 days was significantly enhanced for both G1 phase and S phase cell pellets versus asynchronous cell pellets. In an aspect, the enhancement for both G1 phase and S phase cell pellets versus asynchronous cell pellets is about 20%, about 30%, about 40%, about 50%, about 60%, about 70%, or about 80%.

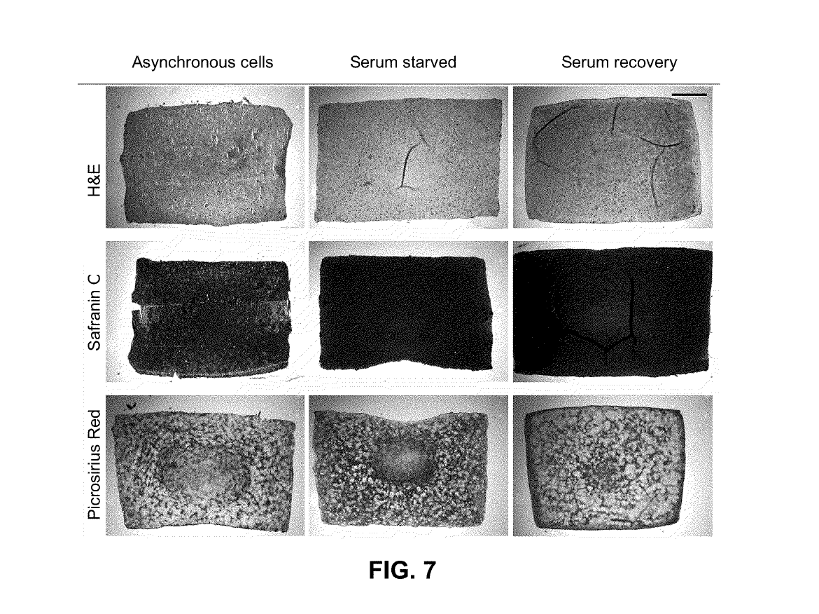

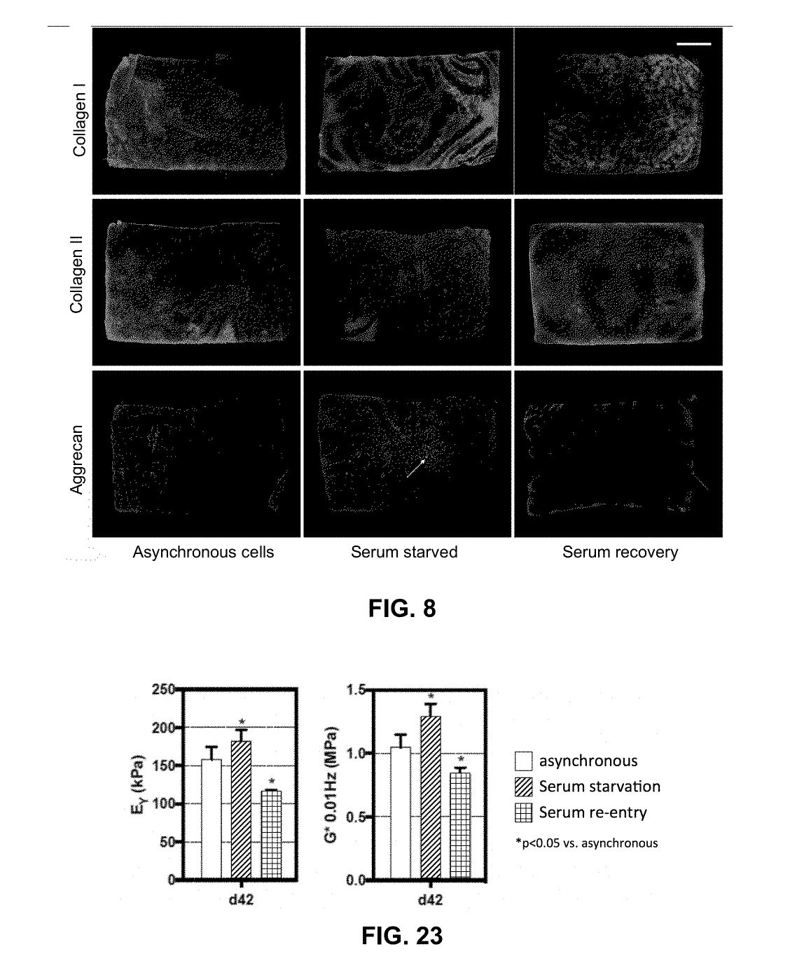

In an aspect, the disclosure provides for methods wherein mechanical functionality, for example, Young's modulus and/or dynamic modulus, for G1 synchronized cells (for example, by serum starvation) is higher than constructs comprised of asynchronous cells. In another aspect, the disclosure provides for methods wherein mechanical functionality, for example, Young's modulus and/or dynamic modulus, for G1 synchronized cells (for example, by serum starvation) is about 10%, about 20%, about 30% about 40%, about 50%, about 60%, or about 75% higher than constructs comprised of asynchronous cells. Additionally, histological stains reveal more intense staining for safranin O (stains proteoglycan present in cartilaginous tissue) for synchronized cells (FIG. 7), suggesting that the type of GAGs produced by the cells may be altered in these populations. For example, examination of the immunohistochemical stains for aggrecan for serum-starved cells indicate more intense staining in the pericellular matrix of each cell (see, for example, FIG. 8).

In another aspect, serum starvation reentry and for example, methylcellulose reentry, include a similar proportion of cells in G1, for example, no more than a 1%, 2%, 3, 4%, 5%, 10%, or 15% difference, but a higher percentage of cells in S, for example, 2%, 3%, 4%, 5%, 10%, 20%, 30% or more, associated with either serum starvation reentry or methylcellulose reentry. In an aspect, this difference of cell proportions in G1 verses S cell phases suggests that it may be this S population of cells that is causing a differentiated response.

One or more embodiments of the disclosed subject matter can generate uniformly differentiating progenitor cell populations. In particular, the cell cycle may be as influential in signaling cell fate as chemical and 3D cues. Thus, in embodiments, the cell cycles of unlimited quantities of cells can be tightly synchronized without the use of one or more active agents or drugs. This can permit synchronous differentiation. Embodiments of the disclosed subject matter can also impact translational advances, e.g., personalized tissue repair. For example, terminally-differentiated or progenitor cells can be removed from a patient, expanded, synchronized, and either returned directly to the patient or used to engineer a tissue graft in which uniform cell differentiation ensures restoration of tissue function.

As noted above, cell-based strategies aimed at tissue repair and fabrication of tissue substitutes require clinically relevant cell numbers that necessitate cell expansion and passaging. There is a tradeoff between multiple passages to attain a sufficient cell number verses desirable tissue properties. In an aspect, the disclosure provides for systems and methods wherein tissue properties are maintained even in the circumstance where multiple passages are employed, for example, 2, 3, 4, 5, 6, 7, or 8 or more passages in order to obtain an ample number of cells based on the desired application. This is surprising especially given the expected inverse relationship between passage number and tissue quality. That is, it would be expected that tissue quality as well as associated properties described herein would decrease with increasing passages in order to obtain additional cell numbers.

In an aspect, cells described herein undergo 1, 2, 3, 4, 5, 6, 7, or 8 expansion passages. In another aspect, the expanded cell properties do not decline after 4, 5, 6, 7, and/8 expansion passages as compared to only 1, 2, or 3 cell expansion passages. In an aspect, the expanded cell properties improve, for example, by about 2%, about 5%, about 10%, about 15%, or about 25% or more after 4, 5, 6, 7, and/8 expansion passages as compared to only 1, 2, or 3 cell expansion passages. In an aspect, the improved properties are selected from ones described herein, for example, the functional response of tissues when exposed to a load, Young's modulus, dynamic modulus, as well as GAG or collagen production.

Cell expansion can be facilitated in two-dimensional (2D) culture, providing opportunities for “cell priming”, where physical and chemical cues that can influence subsequent behavior in three-dimensional (3D) culture are administered. Unfortunately, native terminally-differentiated cells and both undifferentiated and differentiated mesenchymal stem cells (MSCs) exhibit cell-to-cell variability that represents a significant challenge to their optimization for cell-based therapies, as shown in FIG. 4A. Even induced pluripotent stem cells (iPS), which can serve as a patient-specific, unlimited supply of stem cells are limited by their intrinsic heterogeneity after conversion to MSCs and then specific lineages. In yet another aspect, allogeneic cells, autologous cells, and/or embryonic stem cells (ESCs) may be practiced with the systems and methods described herein.

While some heterogeneity may be ascribed to mixed cell populations (e.g., contaminant cells), even in cases where cells are pre-selected for enrichment of a subpopulation, considerable variation in differentiative potential may remain. Mechanisms that govern cell differentiation include cell cycle stage for permitting cellular responses to differentiative cues. Therefore, some of this cell heterogeneity arises directly and/or indirectly from cells being in different phases of the cell cycle, as illustrated in FIG. 4B. Moreover, cells can more uniformly respond to chemical priming regimens if they are synchronized in a specific phase at which they are competent to respond to differentiative cues.

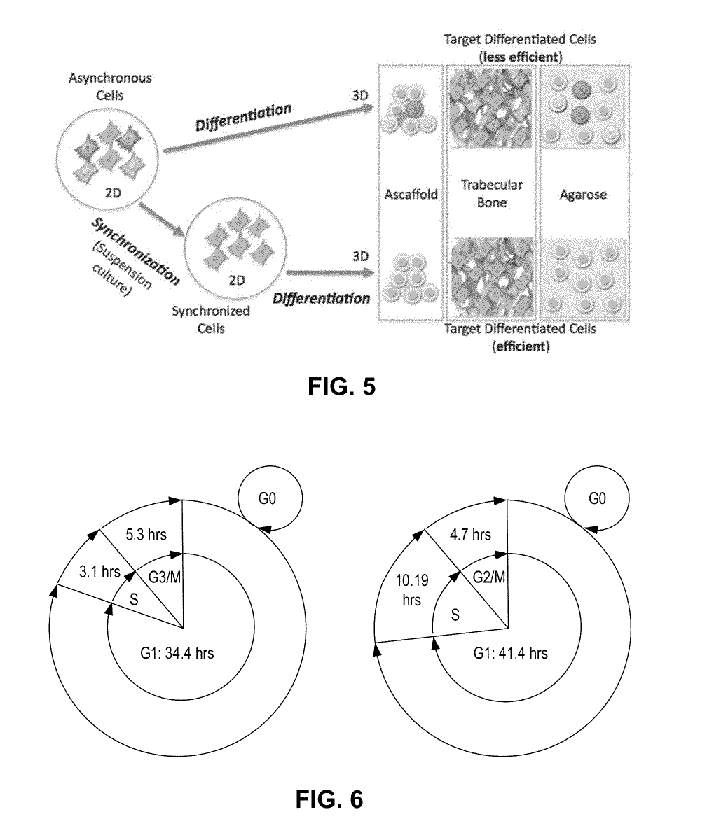

Growth factors, cell priming strategies, and differentiative cues can be markedly more effective if they are administered to cells whose cell cycle synchrony predisposes them to respond to these differentiative cues. In particular, cell synchronization can have a role in 1) modulating chondrogenic differentiation and 3D cartilage tissue development of cells derived from human articular cartilage (chondrocytes), bone marrow (MSCs) and skin (iPS) and 2) modulating osteogenic differentiation and 3D bone tissue development of osteoblasts derived from human bone, bone marrow and skin (FIG. 5).

In an aspect, cells are synchronized using an inert substance, for example a suspension of inert substances. In yet another aspect, cells are synchronized using carbohydrates, polysaccharides, alginate, agarose, and/or alginate bead encapsulation. In another aspect, cells are synchronized using a suspension of compounds or compositions that are capable of inhibiting cells from attaching and/or binding to one another and/or attaching or binding to substrates. In yet another aspect, cells are synchronized using a compound, composition, and/or substance, for example a suspension, which is capable of inhibiting cells from attaching and/or binding to one another such that the cells can be synchronized in a manner that is consistent with the methods and systems described herein.

In an aspect, the chondrocyte cell cycle is synchronized using an agent, compound, or composition described herein. In another aspect, the disclosure provides for cells suspended in an about 0.5% wt/vol, about 1% wt/vol, about 1.5% wt/vol, about 2% wt/vol, about 3% wt/vol, or about 5% wt/vol of a carbohydrates, polysaccharides, alginate, agarose, and/or alginate bead encapsulation solution. In another aspect, the disclosure provides for cells suspended in an about 0.25% wt/vol to about 5% wt/vol, about 0.5% wt/vol to about 3% wt/vol, about 1% wt/vol to about 4% wt/vol, or about 1% wt/vol, or about 2.5% wt/vol carbohydrates, polysaccharides, alginate, agarose, and/or alginate bead encapsulation solution. In another aspect, the disclosure provides for cells suspended in an about 0.5% wt/vol, about 1% wt/vol, about 1.5% wt/vol, about 2% wt/vol, about 3% wt/vol, or about 5% wt/vol methylcellulose solution. In another aspect, the disclosure provides for cells suspended in an about 0.25% wt/vol to about 5% wt/vol, about 0.5% wt/vol to about 3% wt/vol, about 1% wt/vol to about 4% wt/vol, or about 1% wt/vol, or about 2.5% wt/vol methylcellulose solution. In another aspect, the chondrocyte cell cycle can be tightly synchronized using methylcellulose suspension, as opposed to the use of deleterious drugs. For example, an arrest in S phase can be optimal for chondrogenesis, whereas G1 has been implicated for neuro and hepatic differentiation.

In another aspect, suspension culture with methylcellulose arrests cells better than without methylcellulose. In another aspect, suspension culture with methylcellulose arrests cells at least about 5%, 10%, 15%, 20%, or 25% better than without methylcellulose.

In an aspect, successful clinical translation of tissue engineering strategies for articular cartilage repair are dependent on, among other things, rapid development of cartilage extracellular matrix (ECM) proteins to impart functionality to the fledgling construct. To expedite and prime harvested cells to produce cartilage-like tissue in 3D culture, a growth factor priming cocktail as disclosed herein can be used, although there may be variability in the rate and degree of ECM production and engineered cartilage properties depending on cell age, species, and donor. As such, additional techniques can be used to prime the cell for enhanced ECM production. Without being bound to theory, in an aspect, it is hypothesized that a homogeneous population of cells in G1 (growth) or S (synthesis) phase can be primed to begin producing matrix immediately once placed in 3D culture due either to rapid protein synthesis or increased DNA replication. Using well-established biological techniques for arresting cells at various phases, the subsequent effect of cell synchrony on tissue development in 3D pellet culture was explored.

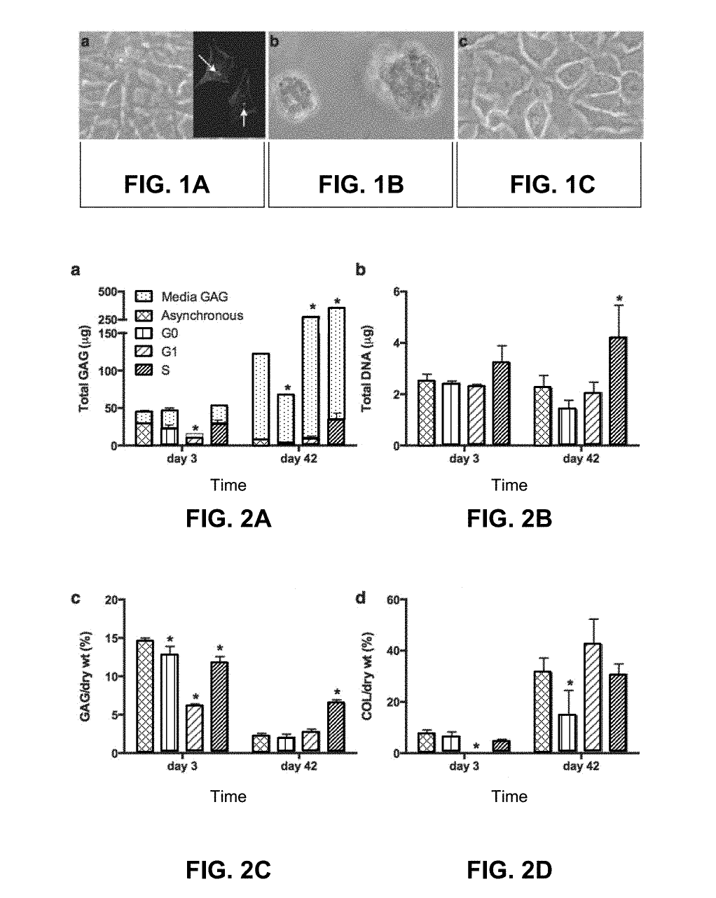

In another aspect, the disclosure provides for a kit comprising, consisting essentially of, or consisting of any of the compounds or compositions disclosed herein. In an aspect, the kit includes any of the combination of compounds or compositions described in Examples 1-3 or FIGS. 1-10. In another aspect, the kit provides for the compositions described in Examples 1-3 or FIGS. 1-10, applied in a manner that is consistent with the methodology of these examples and figures. In another aspect, the kit provides instructions or guidance regarding the use of the compositions or methods described herein.

In an aspect, the kit includes instructions describing the methodology described herein. In another aspect, the kit includes instructions describing the methodology set forth in any of Examples 1-3 or FIGS. 1-10. In an aspect, the instructions are included with the kit, separate from the kit, in the kit, or are included on the kit packaging.

The following examples serve to illustrate certain aspects of the disclosure and are not intended to limit the disclosure.

### EXAMPLES

### Example 1

Methods

Cell Harvest and Extraction: Articular cartilage was harvested from the knee joints of freshly slaughtered 2-4 week old bovine calves (n=4 joints), digested with collagenase IV, and plated at high density in a growth factor cocktail (1 ng/mL TGF-β1, 5 ng/mL bFGF, and 10 ng/mL PDGF-ββ).

Cell Synchronization: At 90% confluence, medium on one subset of cells was switched to serum free medium (chondrogenic medium, CM) to serum-starve the cells for 24 hours prior to use (arresting cells at the G1 checkpoint).

At confluence (FIG. 1A), both asynchronous and G1-serum starved cell populations were trypsinized to create micropellets. A subset of the asynchronous cells was also suspended in methylcellulose (0.1% in DMEM supplemented with 10% FBS, FIG. 1B) for 48 hours to arrest cells in G1 phase. Cells were then extracted from the suspension culture and one subset used to create micropellets while the final subset was plated for 18 hours to allow for cell reattachment and entry into S phase (FIG. 1C), after which micropellets were formed.

Micropellet Culture: A 0.5 mL of a 0.5×10⁶ cell suspension was aliquotted into 1.5 mL sterile screw-top tube, formed into pellets by centrifugation, and cultured for 42 days. Chondrogenic medium was supplemented with 10 ng/mL TGF-β3 (R&D Systems) for the first 14 days and an aliquot of media was saved on each feeding day. At days 3 and 42, micropellet samples were harvested.

Biochemistry: GAG, collagen, and DNA content were determined using the DMMB dye-binding assay, orthohydroxyproline (OHP) assay, and Picogreen dsDNA assay, respectively.

Histology: Acid formalin-fixed samples were paraffin embedded, sectioned (8 μm thick), and stained with Safranin O, Picrosirius Red, and Hematoxylin & Eosin to assess GAG, collagen and cellular distribution, respectively.

Statistics: One-way ANOVA (α=0.05) with Tukey's HSD post-hoc tests was used to compare groups (n=4-5/group) at each time point.

Results

Cell populations synchronized to enter the S phase effected larger cuboidal cells (FIG. 1C) than asynchronous populations (FIG. 1A). Cell synchronization prior to subsequent 3D pellet formation altered the biosynthetic output and content of engineered cartilage. Total GAG retained by the pellet system was significantly reduced as early as day 3 in culture, confirming an altered initial biosynthetic potential. By day 42 of culture, only S phase cells exhibited greater GAG content than asynchronous cells (29.3±4.16 μg vs. 6.37±1.36 μg, p<0.05, FIGS. 2A, 2C).

When GAG lost to the media was taken into account, the total GAG produced over 42 days was significantly enhanced for both G1 phase-methylcellulose and S phase cell pellets versus asynchronous cell pellets (GAG_{G1}=273 μg, GAG_{S}=321 μg vs. GAG_{control}=116 μg, p<0.05). In contrast, G1 cells synchronized by serum starvation exhibited significantly less total GAG production (retained by pellet and present in media, 63.9 μg GAG, p<0.05) than asynchronous cells. DNA content in the asynchronous and G1 phase-synchronized cell pellets remained consistent throughout the culture period (FIG. 2B). S phase cells exhibited an increase in DNA content over the initial DNA content and asynchronous DNA content at day 42 (p<0.05). This indicated cell multiplication over time. Collagen production was consistent across all cell pellet groups except serum starvation (14.7±9.8%/dw vs. 31.5±5.69%/dw, p<0.05, FIG. 2D).

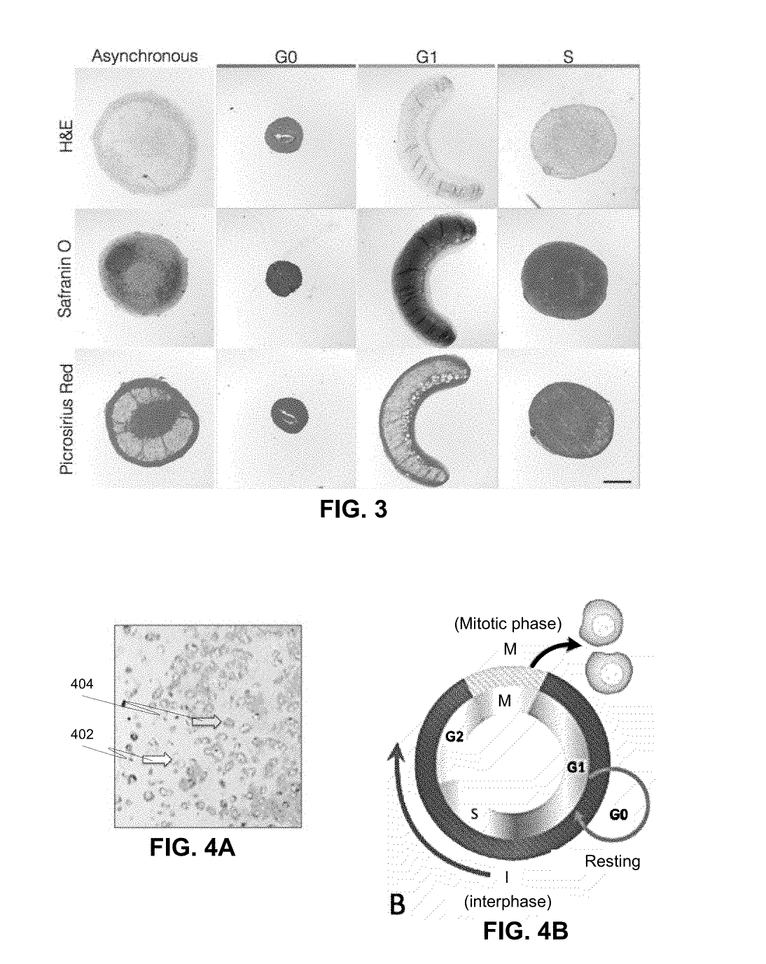

Histological stains for cellularity, GAG and collagen distribution for the various cell phase pellets revealed similar findings (FIG. 3). Asynchronous cell pellets exhibit non-homogeneous distribution of GAG molecules that was countered by intense collagen staining at the periphery and center. Notably, S phase cell pellets revealed the most homogeneous staining for GAG and collagen throughout the entirety of the pellet. In contrast, the shape of the methylcellulose suspended cell pellet reflects non-uniform GAG distribution. Serum starved cell pellets were significantly smaller, with GAG and collagen distribution tightly condensed throughout the pellet.

Control over cell synchrony and specific targeted phase arrests successfully primed cells for 3D pellet culture, eliciting greater biosynthetic activity with accompanying increases in biochemical production, for example, GAG. In particular, pelleting of synchronized cells entering the S phase resulted in homogenous distribution of these matrix molecules across the pellet, for example, reflecting the consistent population of cells primed to undergo DNA replication and protein synthesis.

Embodiments of the disclosed subject matter have the potential to produce superior tissues and to mediate the response of cells to external stimuli. In addition, it has been observed that in asynchronous cells there is a non-homogeneous presentation (length, orientation) of primary cilium (see inset of FIG. 1A). Furthermore, primary cilium can regulate a number of cell signaling pathways, including cytokine-induced pathways such as NF-κβ. Thus, primary cilium can be used to modulate the response via cell synchrony to attain a population of cells resistant to catabolic effects.

Utilizing synchronous cells for engineered cartilage generates superior tissue formation.

### Example 2

Tissue isolation and harvest Articular cartilage was harvested from freshly slaughtered adult canine knee joints, digested in high-glucose Dulbecco's Modified Eagle's Medium (DMEM) with collagenase IV (Worthington Biochemical Corporation, Lakewood, N.J.) for 11 h at 37° C. with shaking. Cell suspensions were filtered through a 70 μm porous mesh and sedimented in a bench-top centrifuge for 15 min at 1500 g. Viable cells were counted with a hemocytometer and trypan blue and plated at high density (20×10³ cells/cm²) in DMEM supplemented with 10% FBS and 1× PSAM. Cells were expanded for one passage before their subsequent use in 3D culture.

Cell synchronization Primary cells following digestion from native tissue were characterized using flow cytometry and found to be predominantly in G1 phase (for example, 95% G1, 4.2% G2, 0.8% S). As such, various techniques were used to synchronize cells to the G1 phase and investigate whether triggered re-entry into the cell cycle is necessary. Flow cytometry was used to determine cell cycle phase time for each cell type (FIG. 6) and characterize the population of cells in each phase (Table 1).

TABLE 1

| | G1 | G2 | S |
|---|---|---|---|
| Asynchronous | 77.38 | 13.88 | 8.74 |
| Serum Starvation | 90.96 | 7.75 | 1.29 |
| Serum Starvation | 82.43 | 11.41 | 6.17 |
| Reentry | | | |
| Methylcellulose | 85.05 | 14.04 | 0.9 |
| Alginate Beads | 84.07 | 14.00 | 1.93 |
| Methylcellulose | 82.61 | 8.14 | 9.25 |
| Reentry | | | |

Two phases of this study were carried out to investigate the various synchronization techniques. Trial 1:
Asynchronous: as a control, one subset of cells were trypsinized at 90% confluence, maintaining an asynchronous population of cells.
Serum-starvation: At 90% confluence, medium on one subset of cells was switched to serum free medium (chondrogenic medium, CM) to serum-starve the cells for 18 hours prior to use. Flow cytometry confirmed that the majority of cells were synchronized to the G1 phase. These cells were then split into two groups, with half of the cells seeded into agarose gels.
Re-entry into the cell cycle after serum-starvation: The other half of the serum-starved cells were re-exposed to FBS-containing medium to encourage cells to re-enter the cell cycle and pass through G1 phase into S phase. Cells were then trypsinized and seeded into agarose gels.

### Trial 2:

Asynchronous: as a control, one subset of cells were trypsinized at 90% confluence, maintaining an asynchronous population of cells.
Alginate bead suspension: At 90% confluence, another subset of cells was trypsinized and encapsulated in 1% wt/vol alginate beads (seeding density: 4×10⁶ cells/mL), synchronizing the cells in G1. After 48 hours, cells were extracted from the alginate beads with a depolymerization solution (55 mM sodium citrate, 0.15M sodium chloride) and seeded into agarose gels.
Methylcellulose suspension: At 90% confluence, another subset of cells was trypsinized and resuspended in 1% wt/vol methylcellulose solution (seeding density: 1×10⁶ cells/mL) for 48 hours, synchronizing the cells in G1. Cells were then extracted from solution through centrifugation and one set was seeded into agarose gels.
Re-entry into the cell cycle after methylcellulose suspension: The other half of the methylcellulose-suspended cells were plated at low seeding density in FBS-containing medium to trigger re-entry into the cell cycle and enter the S phase. Cells were then trypsinized and seeded into agarose gels.

Construct fabrication and culture At confluence, cells were trypsinized, resuspended (60×10⁶ cells/mL), and mixed in equal parts with 4% low-gelling agarose (type VII, Sigma) at 37° C. The chondrocyte/agarose mixture was cast into slabs and cores were produced using a sterile, disposable biopsy punch (Miltex) to yield final dimensions (4 mm×2.34 mm thick). Constructs were cultured in DMEM supplemented with 1× penicillin, streptomycin, fungizone (PSF, Sigma), 40 μg/mL L-proline, 100 μg/mL sodium pyruvate, and 1× ITS premix (insulin, human transferrin, and selenous acid, Becton Dickinson, Franklin Lakes, N.J.). Medium was freshly supplemented with 50 μg/mL ascorbate 2-phosphate, 10 ng/mL TGFβ-3 (Invitrogen), and 0.1 μM dexamethasone (Sigma) and changed every other day.

Mechanical Testing Constructs were tested for their equilibrium E_{Y} and dynamic modulus (G*) in unconfined compression using a custom computer-controlled system. An initial 0.02 N tare load was applied, followed by compression to 10% strain, at a strain rate of 0.05% s⁻¹. After stress relaxation was achieved, a 2% peak-to-peak strain was superimposed at 0.01 Hz.

Biochemistry After material testing, half of the construct was dried and digested in proteinase K solution overnight at 56° C. and the other half was preserved for histology (see below). The biochemical content of each sample was assessed by measuring the sample wet weight, lyophilizing, and then measuring the dry weight. Following digestion, one aliquot was analyzed for GAG content via the 1,9 dimethylmethylene blue dye-binding assay. A second aliquot was hydrolyzed with 12 N HCl at 110° C. for 16 h, dried, and resuspended in assay buffer. Orthohydroxyproline (OHP) content was determined using a colorimetric assay via a reaction with chloramine T and dimethylaminobenzaldehyde, scaled for microplates. Overall collagen content was calculated using a 1:7.64 OHP-to-collagen mass ratio. Total double stranded DNA content was assessed by the Picogreen assay, following the manufacturer's standard protocols.

Histology The other half of each sample was fixed in acid-formalin, paraffin embedded, sectioned (8 iim thick), and stained for histology to assess cellular (Hematoxylin & Eosin), proteoglycan (Safranin O), and collagen (Picrosirius Red) distribution and organization. Immunohistochemistry was also performed to assess the development of collagens I and II and aggrecan in constructs.

Statistics Statistical analyses were performed using two-way analysis of variance (ANOVA) with Tukey's Honest Significant Difference post hoc tests (Statistica), with a=0.05 and statistical significance set at p<0.05 to compare groups across day and synchronization method. Data is reported as the mean and standard deviation of 4-5 samples per time point and group.

Results Synchronization of cells to the G1 phase was generally successful across all synchronization techniques (serum starvation, methylcellulose, and alginate bead encapsulation, Table 1). When these cells are triggered to re-enter the cell cycle through the addition of serum (concomitant with attachment to a tissue-culture surface), progress through G1 into S phase was subsequently observed.

### Trial 1

In trial 1 (through terminal time point of 42 days), mechanical functionality (Young's modulus and dynamic modulus) for G1 synchronized cells (by serum starvation) was significantly higher than constructs comprised of asynchronous cells. While parallel trends were not noted in biochemical content (GAG/dw or GAG/DNA), histological stains reveal more intense staining for safranin O (stains proteoglycan present in cartilaginous tissue) for synchronized cells (FIG. 7), suggesting that the type of GAGs produced by the cells may be altered in these populations. Indeed, close examination of the immunohistochemical stains for aggrecan for serum-starved cells indicate more intense staining in the pericellular matrix of each cell (as an example, please refer to the arrow in FIG. 8).

When synchronized cells were triggered to reenter the cell cycle after serum starvation (confirmed by increased % of cells in the S phase), these constructs exhibited decreased mechanical properties that was accompanied by a loss of glycosaminoglycans GAG (normalized to dry weight) that was not apparent through bulk histological stains (FIG. 7). A poor correlation between quantifiable biochemical constituents (sulfated glycosaminoglycans) and histological staining (visualization of proteolgycans) was noted.

### Trial 2

In trial 2 of the study, alternative methods of G1 synchronization through suspension culture were assessed. In an aspect, it is observed that differential increases in mechanical properties for constructs composed of alginate bead encapsulated cells and constructs for which methylcellulose-synchronized cells were triggered to re-enter the cell cycle.

### Example 3

### Tissue Isolation and Harvest

Human bMSCs were isolated from fresh unprocessed bone marrow (Lonza) of a 22 year-old male donor. Following separation via Percoll gradient, mononucleated cells were plated (5×10³ cells/cm²); adhered MSCs were expanded until passage 4 (passaging 4 times), as described for canine chondrocytes.

### Cell Synchronization

Flow cytometry was used to determine cell cycle phase time for human mesenchymal stem cells and characterize the population of cells in each phase for each synchronization technique described below (Table 2). Table 2 represents percent of human mesenchymal stem cells in each cell cycle phase as determined by flow cytometry.

TABLE 2

| | G1 | G2 | S |
|---|---|---|---|
| Asynchronous | 73.54 | 8.35 | 18.10 |
| Methylcellulose | 94.52 | 1.63 | 3.84 |
| Alginate Beads | 82.48 | 17.52 | 0.00 |
| Methylcellulose | 79.30 | 11.21 | 9.49 |
| Reentry | | | |
| Alginate Beads | 77.47 | 8.86 | 13.67 |
| Reentry | | | |

Asynchronous: as a control, one subset of cells were trypsinized at 90% confluence, maintaining an asynchronous population of cells.
Alginate bead encapsulation: At 90% confluence, another subset of cells was trypsinized and encapsulated in 1% wt/vol alginate beads (seeding density: 4×10⁶ cells/mL), synchronizing the cells in G1. After 48 hours, cells were extracted from the alginate beads with a depolymerization solution (55 mM sodium citrate, 0.15M sodium chloride) and one set was used to create micropellets.
Re-entry into the cell cycle after alginate bead suspension: The other half of the alginate bead-suspended cells were plated at low seeding density in FBS-containing medium to trigger re-entry into the cell cycle and enter the S phase. Cells were then trypsinized and used to create micropellets.
Methylcellulose suspension: At 90% confluence, another subset of cells was trypsinized and resuspended in 1% wt/vol methylcellulose solution (seeding density: 1×10⁶ cells/mL) for 48 hours, synchronizing the cells in G1. Cells were then extracted from solution through centrifugation and one set was used to create micropellets.
Re-entry into the cell cycle after methylcellulose suspension: The other half of the methylcellulose-suspended cells were plated at low seeding density in FBS-containing medium to trigger re-entry into the cell cycle and enter the S phase. Cells were then trypsinized and used to create micropellets.

### Micropellet Formation and Culture

Following trypsinization, cells were counted and a desired volume of cell suspension was aliquotted into 1.5 mL sterile screw-top tubes. The tubes were spun in a microcentrifuge at 37° C. and 2500 rpm for 20 minutes to form a visible cell pellet at the base of the tube. Micropellets were stored in an incubator maintained at 37° C. and 5% CO₂ for the duration of the study (42 days). Media was prepared from high-glucose DMEM with the addition of 100 μg/mL sodium pyruvate (Sigma), 50 μg/mL L-proline (Sigma), 1% ITS+premix (Becton Dickinson), and 1% antibiotic-antimycotic (Invitrogen). 50 μg/mL ascorbic acid (Sigma), 10 ng/mL TGF13-3 (R&D Systems), and 0.1 μM dexamethasone was added fresh to the media on each media change day. At days 3 and 28, micropellet samples were harvested. Media was completely removed to be assayed for GAG content.

### Results

Synchronization of cells to the G1 phase in both synchronization techniques (methylcellulose and alginate bead encapsulation, Table 2) led to increased biosynthetic output by the cells (GAG/DNA, FIG. 10, p<0.05). When methylcellulose suspended cells were retriggered to reenter the cell cycle to the S phase, cells were more prolific in GAG production (FIG. 10, p=0.03). In contrast, when alginate bead encapsulated cells were retriggered to enter the cell cycle to the S phase, cells exhibited reduced biosynthetic capacity, similar to asynchronous cells (FIG. 19, p=0.01). The disparity in response of cells once retriggered to enter the cell cycle suggests that the particular technique of synchronization is of importance.

### Further Examples

In further examples the synchronization approach was applied to MSCs isolated from adipose tissue (ADSCs). MSCs are an attractive cell source due to their increased availability and intrinsic ability to differentiate down desired lineages. Their success in tissue engineering, however, has been limited by variation in differentiation potential and proliferation capacity, and there is poor understanding how to select the most potent cells for clinical application. In particular, while ADSCs are among the easiest to extract, previous reports have found inferior potential for proliferation and chondrogenesis. The synchronization method that was successful for 3D chondrogenic differentiation was applied to ADSCs to demonstrate increased utility of these cells for cartilage tissue engineering.

Two sequential studies were performed. In study 1, the ability of the synchronization approach to enhance chondrogenesis of ADSCs was analyzed. It was expected that modulating the % S phase cells directly would have a useful impact on the glycosaminoglycan (GAG) production of these cells. In study 2, the potential role of primary cilia in mounting this chondrogenic response was analyzed. The primary cilium is believed to play a regulatory role in cell cycle progression and is considered a critical effector of cell mechanotransduction and chondrogenesis. For background, mechanosensitive cells sense perturbations that are transduced to biochemical signals that regulate proliferation, differentiation, and gene expression in stem cells. Furthermore, maintenance of the phenotype of chemically induced differentiated MSCs was found to rely on the presence of primary cilia. Cells are synchronized and primary cilia (incidence rate and cilia length) and the subsequent calcium signaling response of these cell populations to fluid shear characterized. It was expected that that synchronized cells would exhibit an increased cilia incidence rate and decreased cilia length due to their advanced position in the cell cycle and that the increase in cilia presence would facilitate a more robust response to external stimuli.

### Methods of Further Examples

Cell isolation and transduction: Following euthanization for other studies (IACUC-University of Florida), adipose tissue was harvested from healthy, skeletally mature horses (2-5 years). ADSCs were isolated and plated for two passages before being transduced with dual gene lentiviral vector construct encoding a secreted metridia luciferase reporter.

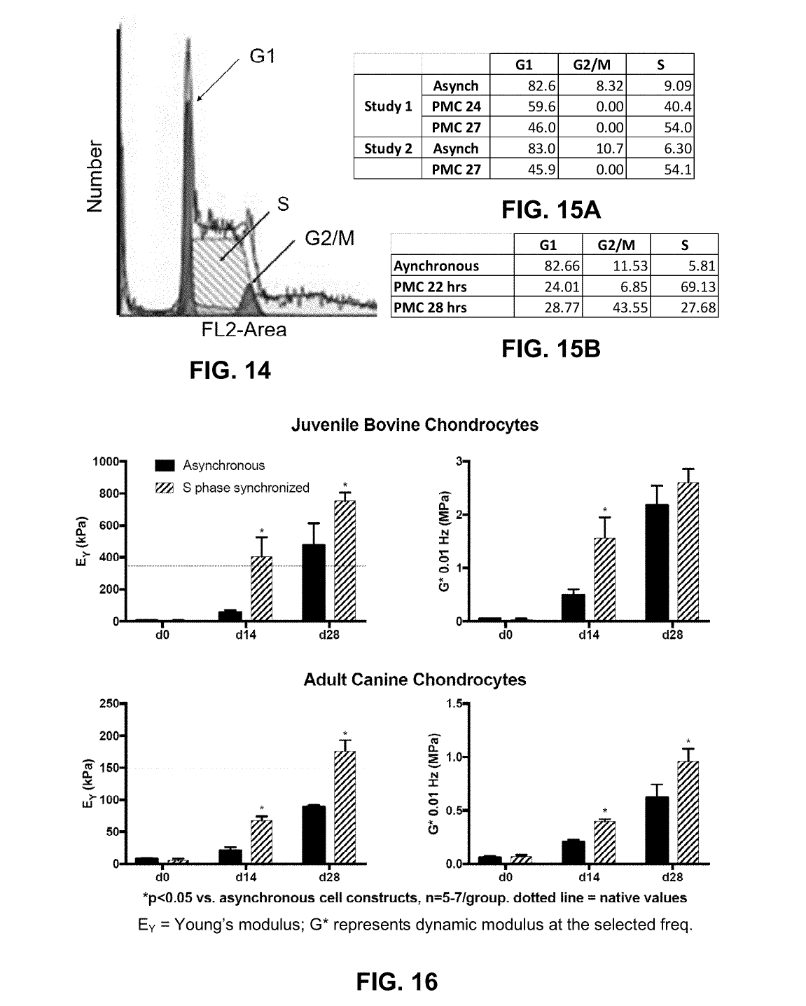

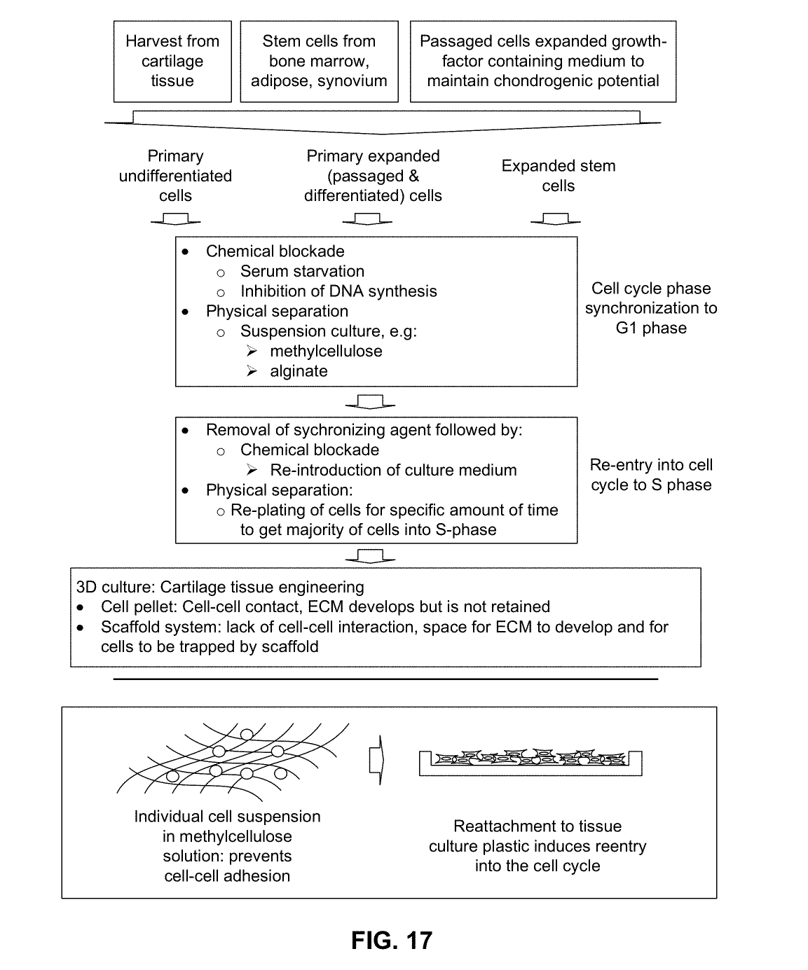

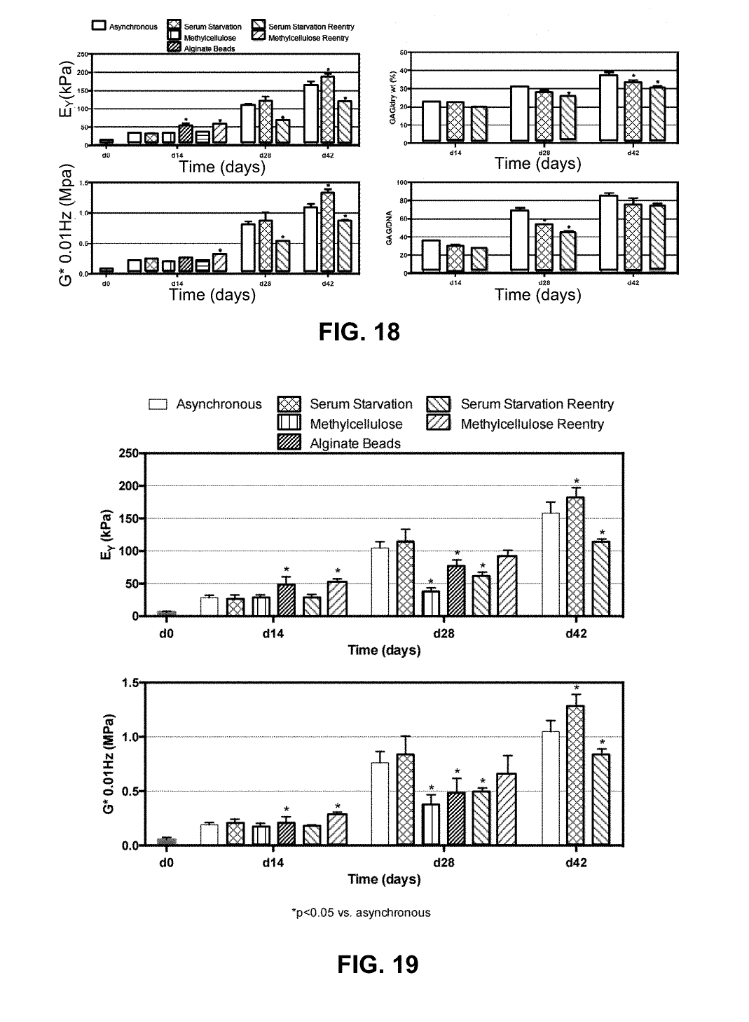

Cell Synchronization Following transduction, cells were returned to monolayer culture for 2 additional passages and used immediately (asynchronous control) or synchronized via methylcellulose suspension and 2D re-plating, as described above. Cell cycle phase was confirmed via flow cytometry (FIG. 14).

Study 1: After 24 hours (post-methylcellulose (PMC) 24) and 27 hours (PMC 27), cells were trypsinized from monolayer culture to produce populations composed of varying % S phase cells. Cells from each group were then used to create cell pellets (0.5×10⁶ cells) and cultured for 35 days in chondrogenic medium supplemented with 10 ng/mL TGF-β3 and 10 ng/mL BMP-6. At days 3, 14, and 35, pellet samples were harvested and evaluated for GAG and DNA content.

Study 2: Cells were re-plated for 27 hours (PMC 27) for cilia staining and Ca²⁺ fluid shear experiments. Cilia staining and imaging: Cells were plated on coverslip-glass bottom dishes and fixed with 4% paraformaldehyde after 27 hours Immunohistochemistry was performed to assess the presence and length of primary cilium (labeled with Alexa-Fluor-488 conjugated acetylated alpha-tubulin and counterstained with TRITC-conjugated phalloidin and diamidino-2-phenylindole (DAPI) for primary cilia, cytoskeleton and nuclear visualization, respectively). Calcium imaging and analysis Cells were plated in silicone wells on glass slides (50×103 cells/well) and imaged after 27 hours for fluid shear response characterization. Briefly, changes in intracellular calcium ([Ca2]i) were tracked using the calcium-sensitive fluorescent indicator Fura Red-AM, and a custom Matlab code was employed to calculate percent responding cells (n=100 cells/slide, pooled across 3 slides/group). Statistics: Two-way ANOVA (α=0.05) with Tukey's HSD post-hoc tests was used to compare groups for biochemical output. Linear regression was performed to assess the relationship between GAG output and % S phase cells. Fisher's Exact Test (p<0.05) was used to assess changes in Ca2+ response and cilia measurements.

### Results

Cell synchronization of equine ADSCs via methylcellulose suspension successfully arrested cells in the G1 phase, and re-plating these synchronized cells triggered cells to re-enter the cell cycle, yielding distinct cell populations with varying composition of % S phase cells (FIG. 15).

Study 1: The biochemical composition of cell pellets was strongly correlated with the % S phase cells. By day 35, PMC 24 cells were slightly more biosynthetically active than asynchronous pellets (GAG/DNA normalized to d3 values, 1.47-fold increase, p=0.32) while PMC 27 cells were significantly more biosynthetically active (2.2-fold increase, p<0.0001, FIG. 12), yielding a strong positive linear relationship between GAG output and % S phase cells (r²=0.8905, FIG. 13).

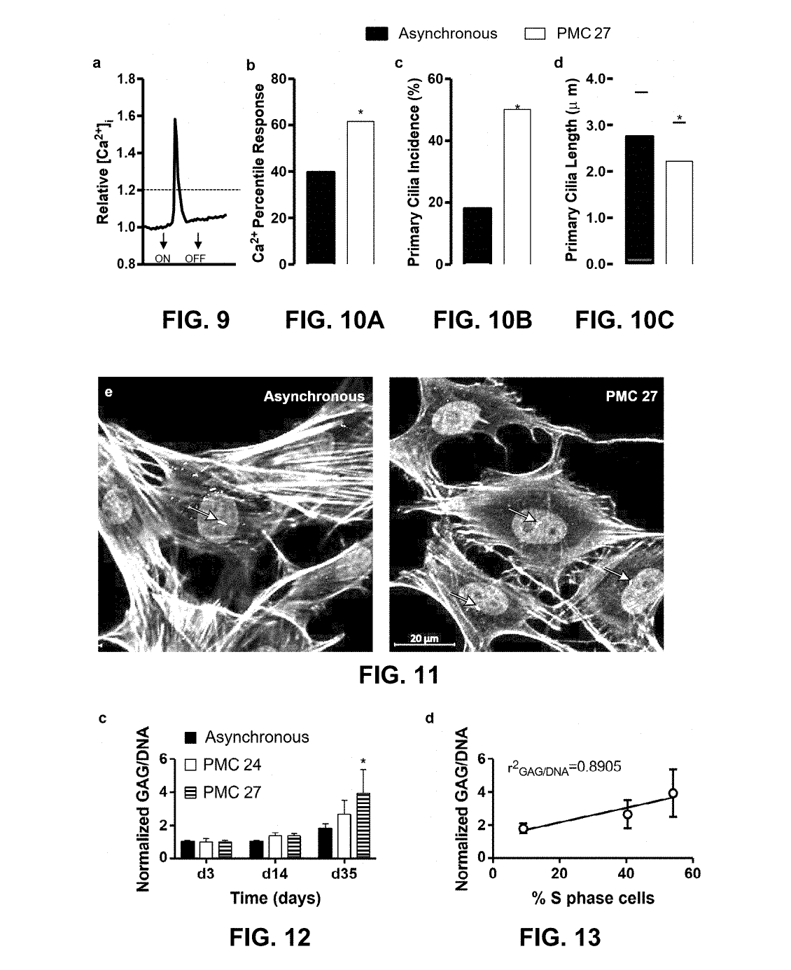

Study 2: The calcium signaling response to fluid shear, as characterized by the increase in intracellular Ca²+ (FIG. 9), was enhanced for synchronized cells; percentile response increased significantly from 40% to 62% (p<0.05, FIG. 10A). Cilia incidence was significantly increased in PMC 27 cells (50% vs. 18%, p<0.05, FIGS. 10B, 12) with commensurate decrease in cilia length (2.22±0.83 μm vs. 2.75±0.94 μm, p<0.05, FIGS. 10C, 11).

The foregoing descriptions apply, in some cases, to examples generated in a laboratory, but these examples can be extended to production techniques. For example, where quantities and techniques apply to the laboratory examples, they should not be understood as limiting.

Features of the disclosed embodiments may be combined, rearranged, omitted, etc., within the scope of the invention to produce additional embodiments. Furthermore, certain features may sometimes be used to advantage without a corresponding use of other features.

FIGS. 10A through 10C show a single cell measurement of intracellular calcium in individual chondrocytes synced to the S-phase with 27 hours of plating in 2D. The percentage of cells responding to an applied physical loading (fluid shear stress) is greater than asynchronous (60% versus 40%) and that there is up regulation of primary cilia (cytoskeletal appendage) that emanates from the cell. That may be a mechanochemical sensor for the cell (arrows in FIG. 11). These data support an example of the biological impact of synchronizing to the S-phase. Note, these calcium studies are performed real-time on a fluorescence microscope using the S-phase cells cultured for 27 hours and still in 3D.

FIG. 12 indicates that leaving the cells in 2D to renter the S-phase for 27 hours is superior to 24 hours.

### Further Examples

As discussed, MSCs are an attractive cell source due to their increased availability and intrinsic ability to differentiate down desired lineages. It has been shown that controlling the cell cycle phase of both terminally differentiated cells and MSCs prior to 3D encapsulation or pelleting yields more chondrogenic tissue. In particular, synchronized cells entering the S phase (synthesis) may be more responsive to exogeneous cues (growth factor stimulation) than cells in G1, G2 (growth), or M (mitosis). The present examples seek to determine if this is due to an increased percentage of proliferating cells (total cells in S, G2, M phases) or a direct consequence of cells residing in the S phase, here we controlled the percentage of cells in each cell cycle phase, varying the % of S phase cells while maintaining a similar overall % of proliferating cells, hypothesizing that increasing the relative proportion of S phase cells in a cell pellet would induce greater biosynthetic output in the presence of chondrogenic factors. We couple this technique with the use of transcriptional reporter systems to better understand the influence of S phase cells on chondrogenic potential.

METHODS: Cell isolation and transduction Following euthanization for other studies (IACUC-University of Florida), adipose tissue was harvested from healthy, skeletally mature horses (2-5 years). MSCs were isolated and plated for two passages before being transduced with dual gene lentiviral report constructs encoding fluorescent reporters. Briefly, the pcDH-GFP lentiviral expression plasmid, encoding tdTomato under constitutive control of a CMV promoter was modified to include GFP under control of a human COL2A1 promoter. Transfection efficiencies were >90% based on flow cytometry. Cell Synchronization Following transduction, cells were returned to monolayer culture for 2 additional passages. At 70% confluence, cells were trypsinized and one subset of cells was used to create 3D cell pellets (asynchronous). Another subset of cells was suspended in 0.5% methylcellulose solution (1M cells/mL) for 48 hours to arrest cells in the G1 phase. Cells were extracted from suspension culture and confirmed to be synchronized to the G1 phase through flow cytometry analysis. Cells were plated to allow for cell reattachment and re-entry into the cell cycle. After 22 hours (post-methylcellulose (PMC) 22) and 28 hours (PMC 28), cells were trypsinized from monolayer culture to produce populations composed of varying % S phase cells. Cells from each group were then used to create cell pellets. Flow Cytometry A subset of cells were fixed, stained, and analyzed on a flow activated cell sorting (FACS) machine to characterize the population of cells in each phase for each synchronization technique. Pellet formation and culture 1.0 mL of a 0.5×10⁶ cell suspension from each group was aliquotted into 1.5 mL sterile screw-top tube, formed into pellets by centrifugation, and cultured for 28 days in chondrogenic medium supplemented with 10 ng/mL TGF-β3. At days 3, 14, and 28, pellet samples were harvested and saved for COL2A1 reporter expression imaging or biochemical and histological analysis. Florescence microscopy GFP and tdTomato expression was characterized by confocal microscopy (Leica LSM 700). Biochemistry:

GAG, collagen, and DNA content were quantified. Histology Acid formalin-fixed samples were embedded, sectioned (5 μm), and stained with Safranin O, Picrosirius Red, and Hematoxylin & Eosin to assess GAG, collagen and cellular distribution, respectively. Statistics Two-way ANOVA (α=0.05) with Tukey's HSD post-hoc tests was used to compare groups. Linear regression was performed to assess the relationship between % S phase cells and % proliferating cells vs. biosynthetic output.

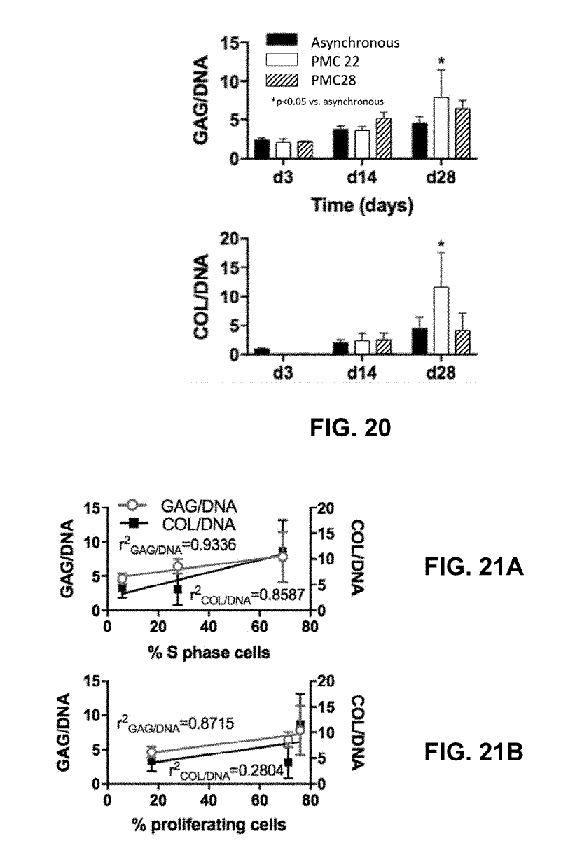

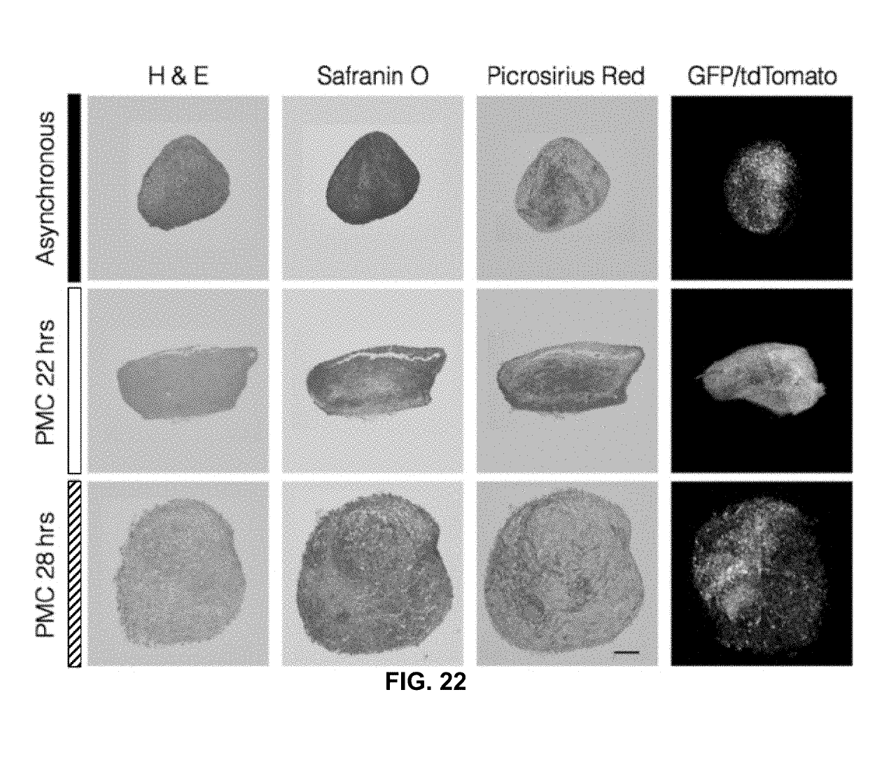

Cell synchronization via methylcellulose suspension successfully arrested cells in the G1 phase and modulating the length of replating time significantly altered the percentage of S phase cells (asynchronous: ˜6%, PMC 22: ˜69%, PMC 28: ˜28%, FIG. 14, 15B). As a consequence, the variation in population composition of the various cell pellets directly influenced the biochemical synthetic output of the cells. By day 28, PMC22 cells were more biosynthetically active, producing 1.7× more GAG/DNA and 2.6× COL/DNA than asynchronous cells and 1.2× more GAG/DNA and 2.8× more COL/DNA than PMC28 cells (p<0.05, FIG. 20). Furthermore, the positive linear relationship between biosynthetic output and % S phase cells was found to be strong for both GAG/DNA and COL/DNA (r²GAG/DNA=0.9336, r²COL/DNA=0.8587, FIG. 21A). In contrast, the relationship between biosynthetic output and total % proliferating cells was found to vary depending on the biochemical marker. While there was a strong positive relationship for GAG/DNA, there was a weak relationship for COL/DNA (FIG. 21B). The modulation of cell cycle phase correspondingly produced varying changes in histological staining and COL2A1 reporter expression in the various groups. Notably, GFP expression was found to be inhomogeneously distributed for both asynchronous and PMC 28 pellets, corresponding to streaky collagen staining throughout the cell pellet (FIG. 22). In contrast, GFP expression for PMC 22 pellets was much more uniform and intense across the sample, paralleling the increased intense collagen staining

While the total percentage of proliferating cells was approximately constant for synchronized cells, increased chondrogenic potential was observed when there was an increase of S phase cells (˜2.5× greater in PMC 22 vs. PMC 28), confirming our hypothesis that priming cells to reside in their S phase prior to pelleting leads to enhanced cartilage-like tissue. Future studies will examine the differential response of S phase cell pellets to additional physiologic stimuli (e.g. osmotic loading, hydrostatic pressure, growth factors and cytokines) to further elucidate the translational potential of synchronized cell populations in repair tissue.

Modulating the composition of cell pellets by controlling the % of cells residing in each cell cycle phase has a direct effect on the differentiation of equine adipose MSCs to produce cartilage-like tissues. Increasing the % of S phase cells in the cell pellet has the potential to yield enhanced chondrogenic tissues and may provide a technique for selecting the most potent cells for clinical application.

According to embodiments, the disclosed subject matter includes method for synchronizing cell cycles of a plurality of cells. The synchronizing may include suspending the cells in an agent and the suspended cells may be serum-starved or treated with a physical agent to synchronize the phase. The cells may comprise stem cells which may be mesenchymal stem cells or patient-specific pluripotent stem cells. The cells may comprise undifferentiated cells. The method may include priming the cells in the S-phase of their respective cycles. The priming may include chemical and/or 3-D priming The priming may be such that a uniformly differentiated progenitor cell population is formed. In the method, prior to the synchronizing, terminally differentiated or progenitor cells may be removed from a patient. After the removing and before the synchronizing the cells from the patient may be expanded.

After the synchronizing the cells to the patient may be returned to the patient.

The method may include using the synchronized cells to engineer a tissue graft.

The synchronizing may include suspending the cells in an agent and wherein the agent comprises a carbohydrate, substance that is inert to cells, methylcellulose. The method may include pelleting of S-phase synchronized cells so as to produce a homogeneous distribution across each pellet. The synchronizing may include suspending the cells in methylcellulose in an about 0.5% wt/vol, about 1% wt/vol, about 1.5% wt/vol, about 2% wt/vol, about 3% wt/vol, or about 5% wt/vol methylcellulose solution. The synchronizing may include suspending the cells in methylcellulose in an about 0.5% wt/vol to about 5% wt/vol, about 1% wt/vol to about 4% wt/vol, or about 1% wt/vol to about 2.5% wt/vol methylcellulose solution.

According to further embodiments, the disclosed subject matter includes a method of engineering tissue. The method includes synchronizing cell cycles of a plurality of cells. The synchronizing may include suspending the cells in an agent. The suspended cells may be serum-starved and the engineered tissue may be used in a tissue graft and/or returned to a patient. The method may include isolating or harvesting tissue from a patient and/or animal. The tissue may be isolated or harvested from cartilage, bone marrow, human bone marrow, and/or cells derived from the synovial lining The tissue may be isolated or harvested tissue from cartilage. The cartilage may be articular cartilage. The engineered tissue may be used in a tissue graft. The engineered tissue may be returned to a patient. The plurality of cells may be selected from the group consisting of stem cells, allogeneic cells, and autologous cells. The stem cells may be selected from the group consisting of mesenchymal stem cells, pluripotent stem cells, and embryonic stem cells. The plurality of cells may be selected from the group consisting of stem cells, allogeneic cells, and autologous cells. The stem cells may be selected from the group consisting of mesenchymal stem cells, pluripotent stem cells, and embryonic stem cells.

It is, thus, apparent that there is provided, in accordance with the present disclosure, tissue engineering methods devices and systems. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicants intend to embrace all such alternatives, modifications, equivalents, and variations that are within the spirit and scope of the present invention.

1. A method of tissue engineering, comprising
obtaining a sample of living cells;
synchronizing the cells to produce a majority concentration in the S phase;
seeding the synchronized cells in a 3D culture to produce functional tissue.

2. The method of claim 1, wherein the living cells are stem cells or tissue cells.

3. The method of claim 1, wherein the synchronizing step comprises synchronizing the cells to a majority G1 phase, followed by re-entry of the cells into S phase.

4. A method of culturing an engineered tissue, comprising:
providing cells in a synchronizing culture that permits cell cycle synchronization;
transferring said cells to a differentiating environment at a time responsive to an estimated phase of the synchronized cell cycle of said first culture.

5. The method of claim 4, wherein the estimated phase is at or near the S-phase.

6. The method of claim 4, wherein the synchronizing culture expands the population of said cells.

7. The method of claim 4, wherein the synchronizing culture is a two-dimensional culture.

8. The method of claim 4, wherein the differentiating environment includes a three-dimensional culture.

9. A method for tissue processing, comprising:
expanding cells;
the expanding including synchronizing and passaging the cells to produce a cell population with a majority concentration of cells in the S phase;
placing the cell population in a chondrogenic environment.

10. The method of claim 9, further comprising engineering cartilage from said cell population.

11. The method of claim 9, further comprising, prior to the expanding, inhibiting dedifferentiation of the cells by exposing the synchronized cells to an agent selected therefore.

12. The method of claim 9, wherein the differentiating environment includes a three-dimensional culture.

13. A method for tissue processing, comprising:
synchronizing cells;
inhibiting dedifferentiation by exposing the synchronized cells to an agent selected therefore;
priming the cells in a manner that causes a major fraction of the cells to reside in their S phase and placing the cells while in the S phase in a differentiating environment.

14. The method of claim 13, wherein the agent includes a physical agent that causes cells to synchronize by constraining the physical relationship between the cells.

15. The method of claim 13, wherein the agent includes a physical agent that keeps cells away from each other.

16. The method of claim 13, wherein the agent includes a physical agent that includes a scaffold material.
